(19) United States
(12) Patent Application Publication
(10) Pub. No.: US 2015/0342781 A1
(43) Pub. Date: Dec. 3, 2015

(54) APPARATUS FOR CREATING SPLIT INCISIONS IN A NUCLEUS DURING CATARACT SURGERY.

(71) Applicant: Eitan Sobel, Rutland, VT (US)

(72) Inventor: Eitan Sobel, Rutland, VT (US)

(21) Appl. No.: 14/289,998

(22) Filed: May 29, 2014

(51) Int. Cl. A61F 9/007

(52) U.S. Cl. CPC A61F 9/00736

(57) ABSTRACT

A cataract surgical apparatus for creating split-incisions in a nucleus of a lens wherein forces applied on the nucleus by a blade (110) during the incision process are at lease partially counteracted to reduce damage to the zonules. In other embodiments, the apparatus further including a nucleus cracking means (151, 152) so that a combined action of creating split-incisions in the nucleus and cracking of the nucleus is carried out by the same surgical apparatus.

### U.S. PATENT DOCUMENTS

| U.S. Pat. No. 4,428,748 | (Jan. 31, 1984) | Gholam A. Peyman, Notilal |
|---|---|---|
| | | Raichand, Edward J. |
| | | Murray |
| U.S. Pat. No. 4,693,245 | (Sep. 15, 1987) | David S. C. Pao |
| U.S. Pat. No. 4,950,272 | (Aug. 21, 1990) | Heinz J Smirmaul |
| U.S. Pat. No. 5,147,368 | (Sep. 15, 1992) | Alan W. Brown |
| U.S. Pat. No. 5,222,960 | (Jun. 29, 1993) | Brooks J Poley |
| U.S. Pat. No. 5,275,607 | (Jan. 4, 1994) | Thomas Y. Lo, Franklin |
| | | Ta, Tolentino Escorcio, |
| | | Kirk H. Packo |
| U.S. Pat. No. 8,016,843 | (Sep. 13, 2011) | Luis J. Escaf -Ultrasonic |
| | | knife |
| U.S. Pat. No. 8,262,682 | (Sep. 11, 2012) | Kenichi Terao |
| U.S Pat. App. | (Aug. 15, 2013) | Kenichi Terao |
| 20130211414 | | |

### EXISTING SURGICAL DEVICES

Chopper Model No. AE-2515 by ASICO
Pre-Chopper Model No. AE-2521 by ASICO
Pre-Chopper Model No. AE-2522 by ASICO

### Slade/Terao Vertical Nucleus Cracker A-4196 by ASICO

### Ernest Nucleus Cracker K5-7240 by Katena

Ahmed/Hoffman vertical scissors by Microsurgical Technologies (MST)

### Slade Vertical Nucleus Cracker E0740 By Storz Ophthalmic Instruments

### Slade Coaxial Chopper E0741 By Storz Ophthalmic Instruments

### STATEMENT REGARDING FEDERALLY SPONSORED RESEARCH OR DEVELOPMENT

Not Applicable.

### BACKGROUND OF THE INVENTION

The present invention is in the technical field of cataract surgical instruments.

More particularly, the present invention is in the technical field of creating split-incisions in the lens nucleus prior to phacoemulsification.

During cataract surgery the damaged lens is removed from the eye and an artificial lens is implanted. The techniques of cataract removal have evolved over the years.

An old technique of cataract surgery is based on removal of the lens in its entirety through a wide incision made in the sclera at the periphery of the cornea. A special capsular bag wraps around the natural lens, therefore, the capsular bag needs to be opened and the lens is pushed out of the capsular bag. U.S. Pat. No. 4,950,272 (Smirmaul) describes a surgical instrument and method for removing the lens of an eye. The surgical instrument is cased on a counteracting design to grab the lens and to pull it out of its capsular bag as one piece.

Today, the techniques of choice for removal of damaged lenses from the eye are done through a small incision at the periphery of the cornea. The lens is consistent of a central nucleus and a peripheral cortex, therefore, microsurgery is done in two stages. First stage, the nucleus is incised and suctioned out of the capsular bag and then, the second stage; the remaining cortex pieces are removed from the capsular bag. Suctioning the nucleus is commonly done by Phacoemulsification, which uses a phaco ultrasonic device, which delivers energy to emulsify the nucleus and suction the resulting nucleus particles from the eye.

Since the nucleus is too big for the phaco device, the surgeons commonly divide the nucleus into smaller fragments. There are many techniques and methods to accomplish this process. Most of the surgeons are creating split-incisions or grooves. Those split-incisions or grooves are used to crack the nucleus either manually by the surgeon or by a cracker device. While there are many techniques and devices to create split-incisions or grooves in the nucleus, the current invention provides new apparatus and insight to this process.

Creating split-incisions or grooves in the nucleus is a difficult process as the lens is a delicate structure supported by delicate fibers called zonules that support and keep the lens in place. A force applied to the nucleus is transmitted to the zonules, damaging and weakening the zonules support system, resulting in surgical complication such as dropping of the nucleus into the vitreous.

A commonly used technique like “Divide and conquer” uses the phaco ultrasonic energy to create a groove in the nucleus that is cracked at a later stage. The local ultrasonic energy reduces the mechanical power required to create the groove and less force is transmitted to the Zonules. However, the ultrasonic energy is also damaging to other eye structures such as the cornea and may overheat the eye globe.

To minimize the force applied, U.S. Pat. No. 4,428,748 (Peyman, et al.), U.S. Pat. No. 6,592,541 (Badrudin Kurwa), and U.S. Pat. No. 8,016,843 (Escaf.) combined ultrasonic emulsifier and mechanical cutter to minimized the force delivered to the Zonules. Again, the potential adverse effect of overheating is still a major concern of this technique and some force is still transmitted to the zonules.

A mechanical nucleus splitter is described by U.S. Pat. No. 4,693,245 (Pao). Pao describes a device for fragmentation of the nucleus with two counteracting components. An elongated member is inserted through a small incision in the periphery of the cornea and then placed beneath the nucleus. The fragmentation piece operates on the lens after it was pulled out of its natural capsular bag. However, most surgeons prefer to work on the lens within its capsular bag. The fragmentation piece is working on the lens like an axe or a hammer and may require a considerable force that potentially could harm other eye structures like the cornea or the capsular bag.

A classic mechanical technique is a chopper. A Chopper device is a microsurgical blade that cuts through the nucleus and breaks off nuclear material, thereby creating grooves that are used for cracking the nucleus into smaller fragments. Example of a chopper device is Nagahara Phaco Chopper Model No. AE-2515 by ASICO. Unfortunately, the chopper devices apply force on the lens nucleus, which inevitably transmitted to the zonules and weakens the eye structure.

Another mechanical device is a pre-chopper, which uses blades that cuts through the nucleus like a knife without breaking off nuclear material thereby creating narrow split-incisions. Examples of pre-chopper devices are Kammann Prechopper superior and temporal angled AE-2521 and AE-2522 by ASICO. Although pre-chopper devices apply less force on the lens nucleus than choppers, they still transmitted some pressure on the zonules and increase the surgical risk.

It must be stated that naturally surgeons realized the chopping and pre-chopping forces are transmitted to the zonules around the lens, therefore surgeons are using a second device or the tip of the phaco machine to counteract the force applied on the nucleus by the chopper or the pre-chopper. Those manual techniques require much experience and highly developed fine motor skills.

Consequently, it made sense to apply the principal of counteracting forces to the process of generating split-incisions or grooves in the nucleus into a new surgical split-incisions creator apparatus

The principal of counteracting forces in the same device is applied in everyday life. Counteracting forces tend to be more powerful, precise and provide the operator with better control of the process. For examples, scissor is much safer and more precise in cutting objects than a knife and pliers or forceps bend objects in safer manner and more precise than a hammer.

In the ophthalmology field, there are many devices that are using to principal of counteracting forces. For example, U.S. Pat. No. 5,275,607 ((Lo et al.) and Ahmed/Hoffman vertical scissors by Microsurgical Technologies (MST) are basically micro scissors designed to cut tissues and objects such as broken artificial lens in the eye. Those devices were not specifically designed for the process of penetrating and creating split-incisions in a nucleus positioned in a capsular bag.

Another device that uses counteracting forces is a cracker. The cracker is used after creating a split-incision or a grove. Most surgeons crack of the nucleus by using two counteracting tools to push against both sidewalls of a split-incision or a groove. However, many surgeons have adopted the cracker to crack the nucleus. The cracker has two opposing plates capable of forceful bilateral separation from each other. The surgeon insert those opposing side plates into a split-incision or a groove in the nucleus and by forceful opening or separation of the side plates away from each other a counteracting pressure is applied to the both sidewalls of the split-incision or the groove and crack the nucleus.

Examples of crackers are U.S. Pat. No. 5,147,368 (Brown), U.S. Pat. No. 5,222,960 (Poley), Slade/Terao Vertical Nucleus Cracker A-4196 by ASICO and Ernest Nucleus Cracker K5-7240 by Katena. Those crackers are inserted into grooves already created in the nucleus.

Combo devices combine of a chopper device or a pre-chopper device together with a cracker. This combination significantly shortened the surgical process and made it safer. Example of combination of pre-chopper and cracker devices is U.S. Pat. No. 8,262,682 (Terao) and Akahoshi Combo Pre-chopper AE-4284 by ASICO. Regardless, the Akahoshi Combo Pre-chopper still applies pressure that is transmitted to the zonules. In addition, Combo Pre-choppers are suitable to only for stage 2 or 3 cataracts. In cataract stage 4, the nucleus is too hard to be cut by the Akahoshi Combo Pre-chopper. In cataract stage 1, the nucleus is too soft for the small lateral surface area of Akahoshi Combo Pre-chopper and the cracking action is not effective.

U.S Pat. App. 20130211414 (Terao) is another combo device. Teraco created two blades that are used as one for the purpose of cutting the nucleus. In one embodiment the surgeon cuts the nucleus by drawing the instrument toward the surgeon and then separate the two blades. In another embodiment, the instrument is used in a side-to-side motion with the surgeon cutting across the nucleus rather than forming an incision toward the surgeon. Similar devices by Slade are already marketed by Storz Ophthalmic Instruments: Slade Vertical Nucleus Cracker E0740 and Slade Coaxial Chopper E0741. Again, the devices creating the split incisions by created by cutting motions either toward the surgeon or side to side. Cracking of the nucleus is done by rotating the nucleus in 90 degrees and then separating the two cutting blades.

### BRIEF DESCRIPTION OF THE INVENTION

The present invention provides apparatus to perform split-incisions in a nucleus to be used for cracking of the nucleus. The present invention utilizes known counteracting principals and structure of tools that are used in everyday life and in the medical and surgical fields and applies those principals and tools to the process of creating split-incisions in a nucleus and cracking the nucleus during cataract surgery.

However, applying the concept of simple scissor cutting of the lens seems rather complex as the position of the lens in the capsular bag makes accessibility to the lens from two opposing ends rather challenging. Therefore, so far, knife-cutting solutions and ultrasound assisted solutions have been utilized for this purpose.

The split-incision apparatus is specifically designed to penetrate a nucleus positioned in a capsular bag with relative ease. Some embodiments of the split-incision apparatus are designed to further deepen the penetration of the split-incision apparatus in the nucleus with each action. The split-incision apparatus does not require expelling of the lens out of the capsular bag. The split-incision apparatus is designed to cut split-incisions in the nucleus in a safe and controlled manner. Unlike scissors that are relative blunt and cut materials like a guillotine the split-incision apparatus may be used over and over again essentially “digging” a split-incision. The split-incision apparatus is designed to considerably reduce forces transmitted to the zonules therefore, the split-incision apparatus is less likely to cause damage. The split-incision apparatus of the current invention require less experience and less training from the surgeons. The requirements of strong dexterity skills and highly developed fine motor skills are much less crucial with the new split-incision apparatus. The resulting split-incisions enable surgeons to crack the nucleus into smaller fragments either manually or by using a cracker. Some of the embodiments of the new split-incision apparatus combine creating split-incisions or grooves actions with cracking actions in the same apparatus, thereby shortening the surgical process and making it even safer.

The simplest embodiment of the split-incision apparatus incorporates two or more narrow blades. Those blades move toward each other and away from each other. The mechanism of movements varies and in the drawing a cylindrical tracks or a parallel tracks are shown. The narrow blades are in approximately perpendicular position to the track. The handle assembly delivers the necessary force to move the tracks, which control the movements of the narrow blades. The surgeon operating this device simply pushes the narrow blades into the nucleus and then using the handle, the surgeon moves the narrow blades either toward or away from each other, thereby forming a split-incision in the nucleus.

Obviously, blades design shape and sharpness controls the nucleus cutting properties and the nucleus penetration properties of the blades. Different embodiments may use different blades shapes and designs. Some blades are design for cutting along the split incision lines. Other blades may have good penetration properties, but may not cut the nucleus along the split incision lines, therefore, those blades tend to wedge into the nucleus. Some designs include multiple blades like a saw and other designs combine various types of blades to control the movements and the position of each part of the split-incision apparatus relatively to the nucleus.

The movement mechanism is not the essence of the new split-incision apparatus and many mechanisms known to people skilled in the art are available to accomplish this goal. In another embodiment, an additional device is attached to the split-incision apparatus and mechanical power source such as a foot pedal unit transmits movement power to the device. In yet another embodiment, a motorized unit is attached to the split-incision apparatus and assists with the mechanical movements of the counteracting blades.

In another embodiment, the blades are bent toward each other. Bending of the blades split the force applied on the nucleus into several vectors. A vector of the force along the blade further increases the penetration of that blade into the nucleus. Conversely, an embodiment with outward bending of the blades further increases the penetration of the blade into the nucleus when the blades move away from each other. Obviously a combination of inward and outward movement of the blades is possible as well.

Yet, in other embodiments, the opposing blades of the split-incision apparatus are wider and designed to break off material from the nucleus and thereby creating a wide split-incision, or in other words, a groove in the nucleus.

Other blade embodiment is complementary. The male and female designs are more effective in finalizing the cutting action of the blades.

Another complementary embodiment includes two blades creating two roughly parallel narrow incisions in the nucleus. The nucleus material in between the roughly parallel narrow incisions is then scooped by a counteracting wedging blade.

Another embodiment of the device combines both a split-incision apparatus and a cracker. In this embodiment, the counteracting blades move toward each other until those blades are, at least partially, overlapping each other. At this position, those counteracting blades are separated bilaterally to their movement path and are used for a cracking action. The cracking action is accomplished in many different mechanisms. For example: parallel tracks design separates bilaterally apart. In another embodiment, a cylindrical track design separates the opposing blades by rotation of the blades away from each other.

The lateral surface of the blades is relatively small to the side plates of a traditional cracker. Therefore, in other embodiment, larger plates with larger surface areas that separate from each other are combined with the split-incision apparatus. There are several embodiments that accomplish this goal.

In one embodiment the side plates of a cracker are used as the counteracting parts of the split-incision apparatus. At least one counteracting blade is attached to the bottom surface of each of the side plates and sliding movements forward and backward of the side plates relative to each other along the split-incision path create a groove in the nucleus similar to counteracting saws action.

In another embodiment, the cracker portion of the device will serve as one side of the counteracting device. Therefore, the cracker side plates have at least one blade that wedges into the nucleus and stabilizes the cracker relatively to the nucleus. The opposing counteraction is done by at least one blade that will act like a digger and is digging nucleus material under the cracker allowing burying the cracker in the newly formed groove. This action of forward backward movements of the digger is continued until the cracker is buried enough in the nucleus to ensure that the cracker's action of lateral separation is cracking the nucleus.

In yet another embodiment, the split-incisions is replaced by a split-tunnel created by a counteracted hollow blade, or in other words a counteracted needle, inserted inside the nucleus material. A tunnel is created in the nucleus that is used for cracking. The cracking action is performed by a balloon inserted through the hollow needles and the cracking action of the nucleus is done by inflating the balloon like angioplasty balloons.

There are many other embodiments that are possible that are basically a variations of the same concepts. Furthermore, different embodiments may not necessarily provide any advantage or improvements over the embodiments presented in this document, therefore, not all the possible variations are shown in the drawing.

For example: in another embodiment not shown in the drawing, a cracker portion of the embodiment is located above two counteracting blades track. After completing the creation of a split incision, the cracker portion is lowered down into the split-incision just created by the counteracting blades. In yet another embodiment, the cracker portion of the embodiment is again located above the counteracting blades. Then, the apparatus is rotated upside down for working with the cracker portion. In yet another embodiment not shown in the drawing, the cracker portion of the embodiment is located just proximately to the counteracting blades. After completing the creation of a split incision, the side plates of the cracker are pushed forward into the split-incision created by the counteracting blades.

The movement mechanism is not the focus of the new split-incision apparatus and many mechanisms known to the ones skilled in the art are available to accomplish the same goal. The drawings were made as an illustration only and other designs are acceptable.

In another embodiment not shown in the drawing, another device is attached to the split-incision apparatus. Mechanical power source such as a foot pedal unit transmits mechanical power for the movements of the blades. In yet another embodiment, power source and a motorized unit is attached to the split-incision apparatus and powers the movements of the counteracting blades, simplifying the cutting and cracking actions. Alternatively, the attached device propels the cracking action of the side plates.

### BRIEF DESCRIPTION OF THE DRAWINGS

FIG. 1 shows a view of a split-incision apparatus having at least two cutting blades for creating split-incisions in the nucleus. An additional view in the circle demonstrates inward bending the blades

FIG. 2 shows a split-incision apparatus having at least two wider blades for cutting grooves in the nucleus.

FIG. 3 shows a split-incision apparatus having at least two blades with larger sides' surface areas. An additional view in the circle demonstrates separation of the blades for the purpose of nucleus cracking action.

FIG. 4 shows another example of the split-incision apparatus. The blades are separated from each other. The blades move along cylindrical tracks.

FIG. 5 shows the split-incision apparatus of FIG. 4. as the blades close and even overlap each other.

FIG. 6 shows the split-incision apparatus of FIG. 4. as the blades separate by relative counter rotational movements of the cylinders.

FIG. 7 shows a split-incision apparatus with bilateral outward-inwards movement

FIG. 8 shows a split-incision apparatus with two counteracting blades in a male-female configuration.

FIG. 9 shows the split-incision apparatus shown in FIG. 8 as it closes.

FIG. 10 shows a double split-incision blades counteracted by a wedge blade in an open position.

FIG. 11 shows a slightly different embodiment of the split-incision apparatus of FIG. 10 as it closes. This embodiment includes a separation plate 148.

FIG. 12 shows a cracker having two plates for cracking the nucleus and the bottom side of the plates include counteracting saw blades.

FIG. 13 shows the same cracker of FIG. 12 shown where one of the blades shifted forward.

FIG. 14 shows the same cracker of FIG. 12 shown where the other plate now is shifted forward.

FIG. 15 shows the same cracker of FIG. 12 in an open position used for the nucleus cracking action.

FIG. 16 shows a cracker having two plates for cracking the nucleus and the bottom sides of the plates include sets of counteracting bumpy surface capable of scraping the nucleus and creating a groove in the nucleus. One of the plates in FIG. 16 is shifted forward.

FIG. 17 shows the same cracker of FIG. 16 shown where the other plate now is shifted forward.

FIG. 18 shows the same cracker of FIG. 16 in an open position of the nucleus cracking action.

FIG. 19 shows a cracker-digger combination where the cracker portion wedges into the nucleus and a counteracting blade called the digger that digs under the cracker

FIG. 20 shows the same cracker-digger of FIG. 19 where the digger moved forward relatively to the cracker.

FIG. 21 shows the same cracker-digger of FIG. 19 in a superior view showing slight opening of the cracker.

FIG. 22 shows a different design of a cracker-digger in which the digger is pulled back against the cracker blades.

FIG. 23 shows the same cracker-digger of FIG. 22 as the cracker opens for the nucleus cracking action.

FIG. 24 shows a split tunnel maker and a balloon cracker as an hollow blade is pushed against a wedge blade.

FIG. 25 shows the same split tunnel maker and a balloon cracker of FIG. 24 as the hollow needle formed a split tunnel.

FIG. 26 shows the same split tunnel maker and a balloon cracker of FIG. 24 as the hollow needle is pulled out leaving a collapsed balloon the split tunnel.

FIG. 27 shows the same split tunnel maker and a balloon cracker of FIG. 24 as the balloon cracker inflates and crack the nucleus.

### DRAWING REFERENCE NUMERALS

110, 111—Thin blades
110-A, 111-A—Bended thin blades
112—Internal cylinder track
113—External cylinder track
115—Illustration of moving handle
115—Spring
116,117—Wide blades
118—External cylinder track
119—Illustration of moving handle
120,121—Blades
122,123—Flat tracks
124—Illustration of moving handle
125—Illustration of separation handle
126—Separation pin
130,131—Blades
132—Internal cylinder track
133—External cylinder track
134—Illustration of moving handle
135, 136—Counteracting blades
137, 138—Arms for moving blades
140—Female configuration of a blade
141—Male configuration of a blade
142—Internal cylinder track
143—External cylinder track
144—Illustration of moving handle
145, 146—Two blades combination
147—Counteracting wedge blade
148—A distal separation plate
149—A proximal separation plate
150,151—Counteraction saw blades
152—Internal cylinder track
153—External cylinder track
154—Illustration of moving handle
150,151—Counteracting blades
152—Side plate of a cracker
153—Cracker arms
154, 155—Pressing handles for separating side plate of the cracker
156,157—Moving mechanism of side plates
160,161—Counteracting scraping means
162—Cracker arms
163, 164—Pressing handles for separating side plate of the cracker
170—Side plate of a cracker
171, 172—Wedging blades of the cracker
174—The digger
175—The digger handle
176, 177—Pressing handles for separating side plate of a cracker
180, 181—Side plate of a cracker
182, 183—Wedging blades of the cracker
184—The digger
185, 186—Pressing handles for separating side plate of a cracker
187—The digger handle
190—A hollow blade, needle like
191—A wedging blade
192—Operator side of the hollow blade
193—Cracking balloon

### DETAILED DESCRIPTION OF THE DRAWINGS

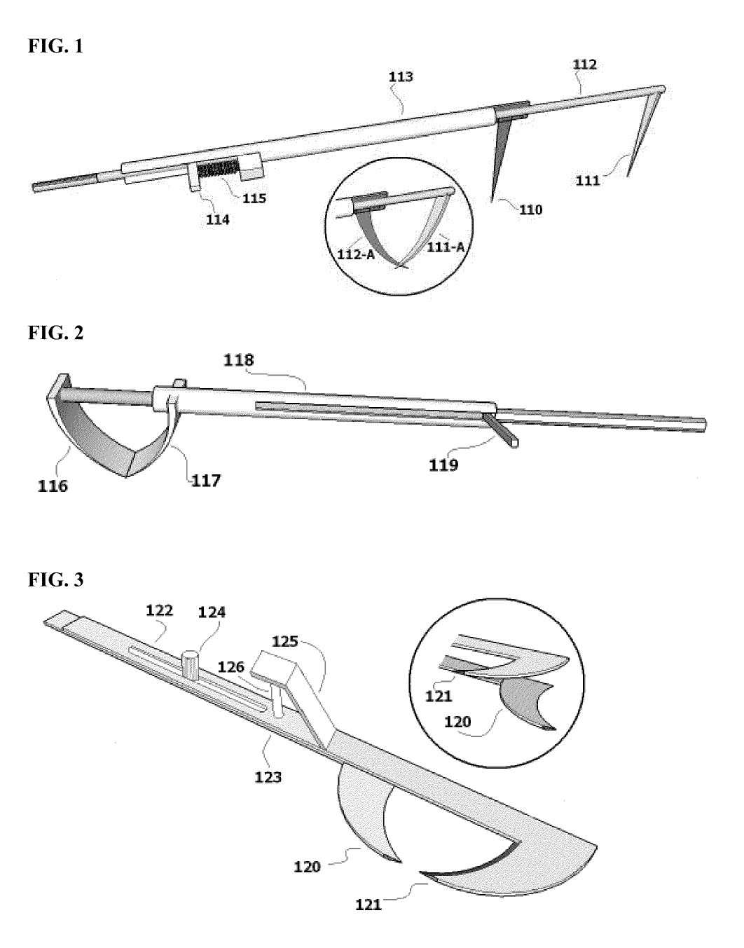

Referring now to FIG. 1, a split-incision apparatus is shown with thin blades 110 and 111, which are easily pushed into the nucleus and controlled by the handle 114 via coaxial tracking system as cylinder 112 moves through cylinder 113. A split line is created by the movement of the two narrow blades 110 and 111 toward each other or away from each other.

Further penetration of the narrow blades into the nucleus is achieved by bending blades 110-A and 111-A toward each other as shown in the circle view. As the blades get nearer each other, they penetrate deeper into the nucleus material.

In alternative embodiments, blades 110 and 111 are replaced with a multitude of blades similar to a saw or a scraper.

In other embodiments, shape modifications of blades such as 110 and 111 affects the penetration or the cutting properties of the blade. For example, a blade with good penetration properties but poor cutting properties is used to stabilize that portion of the device relatively to the nucleus while the counteracting blade or blades cut the nucleus.

The handle assembly portion including handle 114 and spring 115 of FIG. 1 and subsequent figures were drawn as an illustration only as many methods and techniques are available to forcefully move two tracks counter to each other. The handle assembly illustration shown should not constrain to the scope of the invention. In another embodiment, another unit is attached to the split-incision apparatus and mechanical power source such as a foot pedal unit transmits the power of movement to the device. Other embodiments include power sources and motorized unit attached to the split-incision apparatus to assist with the mechanical movements of the counteracting blades. Referring now to FIG. 2, blades 116 and 118 are wider. By moving blades 116 and 118 toward each other a groove is created in the nucleus. The bending of the blades 116 and 118 toward each other enables easy penetration of the blades into the nucleus material.

In a different embodiment, blades 116 and 118 are a multitude of blades similar to a saw or a scraper.

Again, different embodiment, shape modifications of blades 116 and 118 affects the penetration or the cutting properties of the blade. Blades with good penetration but poor cutting properties wedge into the nucleus and stabilize a portion of the device in relation to the nucleus while the counteracting blade or blades cut the nucleus.

Again, the handle assembly portion including handle 119 of FIG. 2 and subsequent figures is shown as an illustration only as there are many methods and techniques to forcefully move two components counter to each other. The handle assembly illustration shown should not constrain to the scope of the invention.

Referring now to FIG. 3, blades 120 and 121 have larger side surface areas. The handle assembly portion shows parallel tracks. Movements of handle 124 will be transmitted to blades 120 and 121 and move them toward and away from each other. Additional mechanisms is provided in this illustration such as handle 125 that using pin 126 to push plate 123 away so that the two blades 120 and 121 are separated as shown in the circle view, thereby using the lateral surfaces of the blades for the nucleus cracking action.

Again, the handle assembly portion including handle 124, 125 and 126 of FIG. 3 are shown as an illustration only as there are many methods and techniques to forcefully separate two components counter to each other.

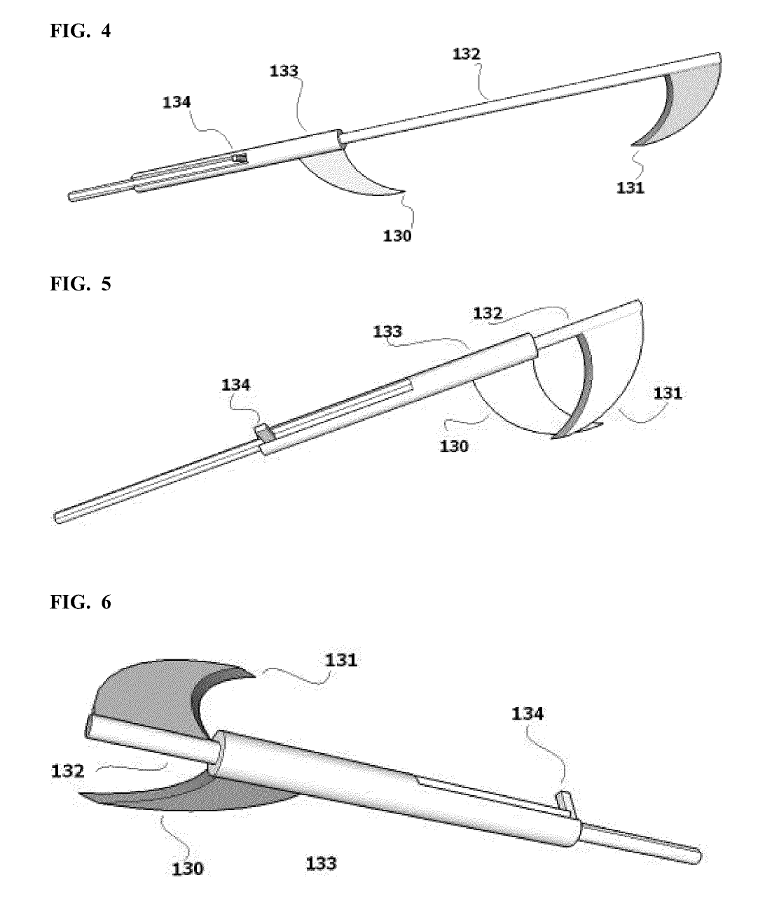

Referring now to FIGS. 4, 5 and 6, the drawing show a simple illustration of one of many mechanism that is used to separate blades 130 and 131 for nucleus cracking action. Blades 130 and 131 are moving along a cylindrical tracking system in which cylinder 132 is moving in cylinder 133. FIG. 5 shows the two blades 130 and 131 reaching each other and later overlapping each other. In FIG. 6, blades 130 and 131 are rotating in opposite direction using the coaxial tracking system, thereby separating from each other and acting as a cracker. Again, the handle assembly portion including handle 134 is shown as an illustration only. Moving handle 134 back and forth will control the cutting movement of blades 130 and 131. Rotation of handle 134 will separate blades 130 and 131 as shown in FIG. 6 for the cracking action of the split-incision apparatus.

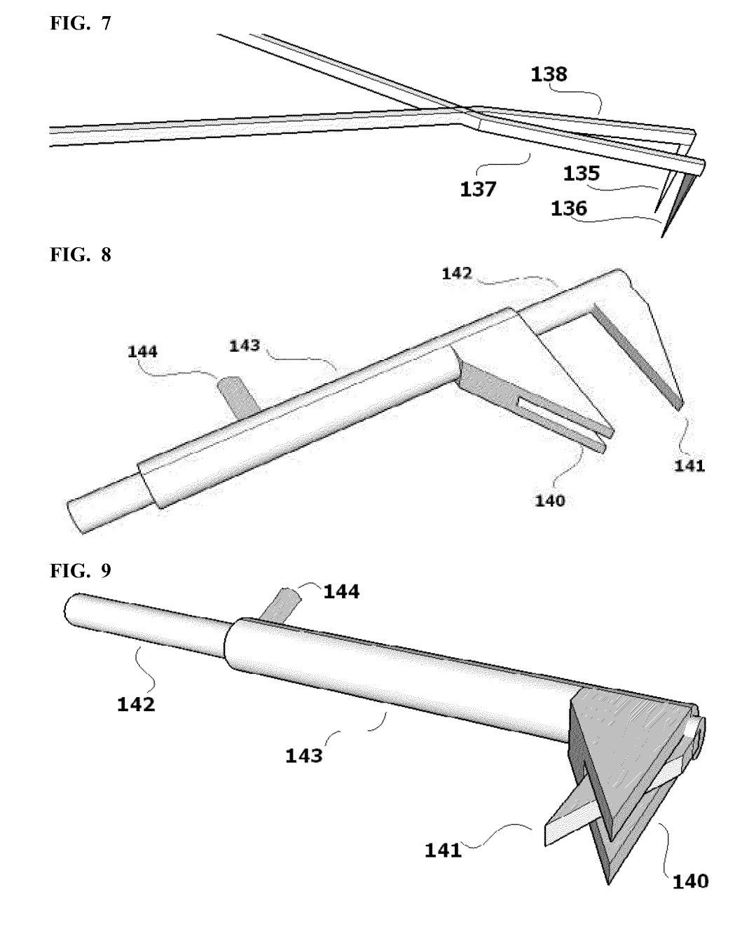

Referring now to FIG. 7, a split-incision apparatus is illustrated having two arms 137 and 138 for moving the blades 136 and 135 respectively along the cutting path thereby creating a split-incision in the nucleus. The split-incision apparatus of FIG. 7 is different than the other split-incision apparatus as its cutting path is about perpendicular to the long axis of the split-incision apparatus.

Referring now to FIGS. 8 and 9, the blades 140 and 141 are complementary male and female design. This design might improve the cutting qualities of the blades. Many other complementary designs are possible and this drawing was created as an illustration only.

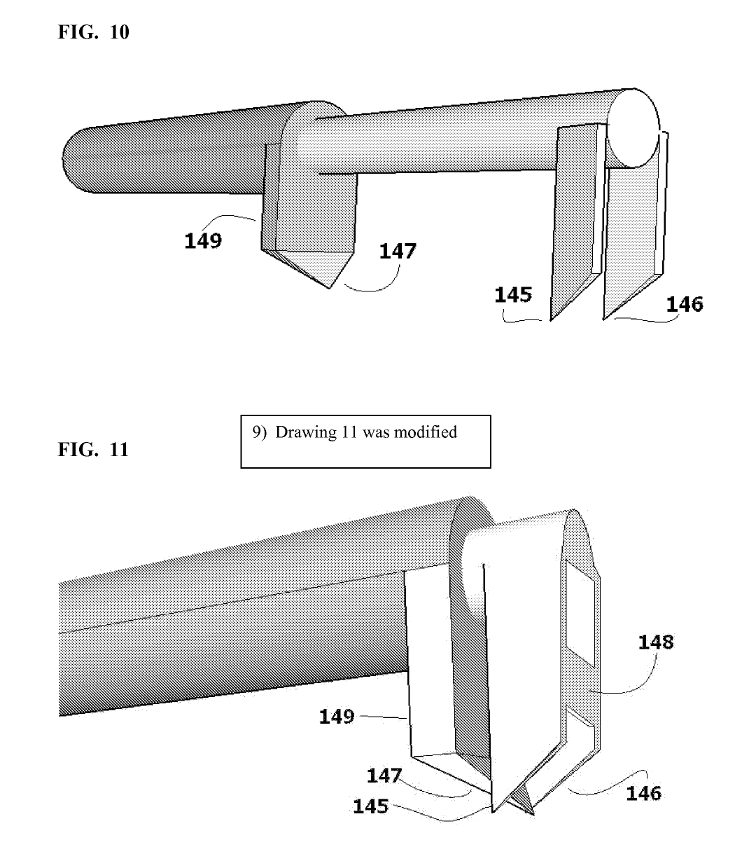

Referring now to FIG. 10, blades 145 and 146 are used to create two roughly parallel incisions in the nucleus. The material of nucleus between the two incisions created by blades 146 and 146 is removed by scraping action of wedging blade 147 that is pushed forward and scrapes the nucleus material between the two incisions. FIG. 11 shows a similar embodiment as it closes. The same apparatus is also used for cracking of the nucleus by rotating the nucleus by 90 degrees and then by opening the device, the proximal plate 149 separate from the back side of blades 145 and 146 and is used for cracking the nucleus. In FIG. 11 an additional distal plate 148 is added to increase the surface area of the distal back side of blades 145 and 146.

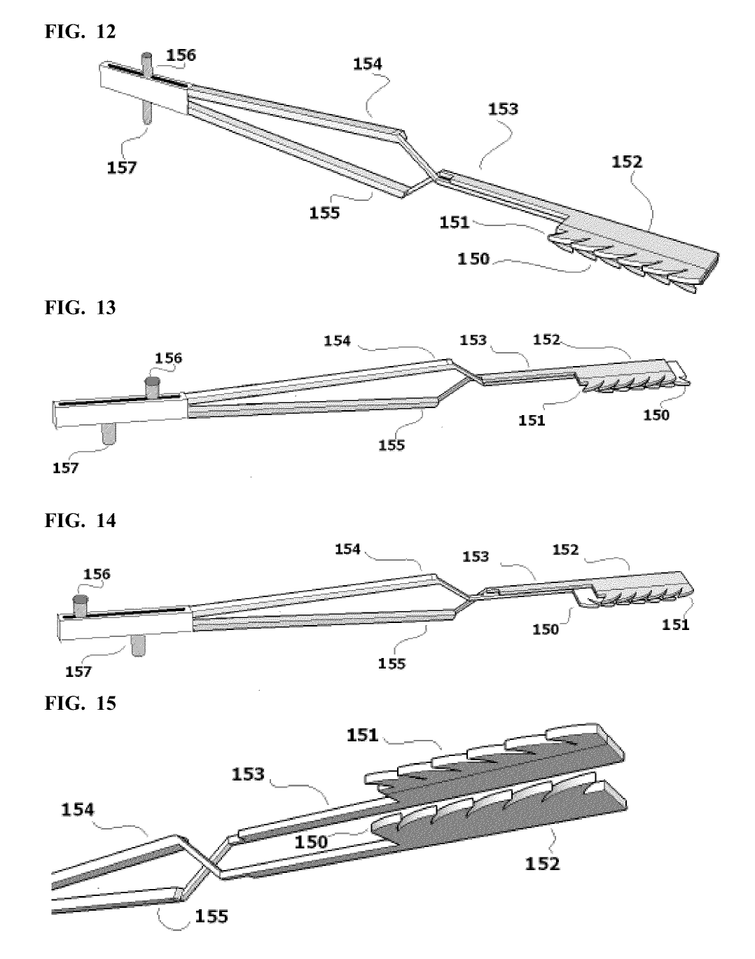

Referring now to FIGS. 12, 13,14 and 15, the split-incision apparatus shown is build on the basis of a cracker presented in drawing having two opposing side plates. The bottom of the plates 150 and 151 incorporate counteracting saws that penetrate the nucleus. The drawings demonstrate the slight movement forward backward of the counteracting saw blades. In FIG. 13, saw 150 is in front of saw 151. In FIG. 14, saw 151 is in front of saw 150. This back and forth movement digs a groove for the cracker to be buried in the nucleus material. Eventually, in FIG. 15 the cracker opens up against the sidewalls of the groove created by the counteracting saws and cracks the nucleus. FIG. 15 shows an illustration of the cracking technique achieved by squeezing points 154 against point 155 As stated before, there are many embodiments for forcefully move two tracks counter to each other and many embodiments for the separation of the side as well as additional manual and motorized attachments known to the ones skilled in the art. The handle assembly portion including handle 156, 157 and 154, 155 are shown as an illustration only.

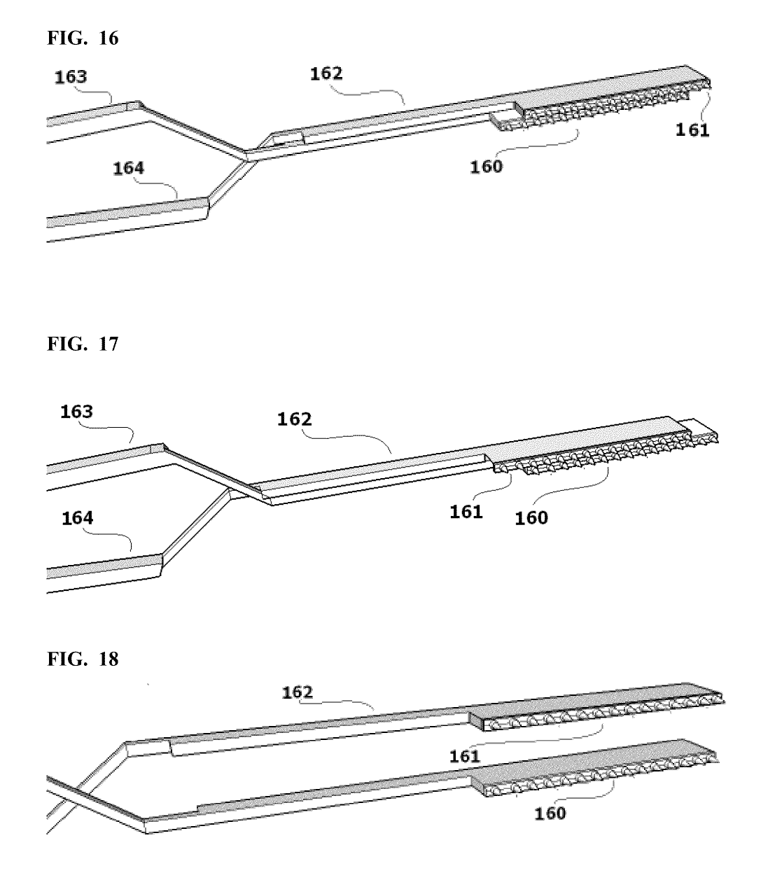

Referring now to FIGS. 16, 17 and 18, the cracker action are essentially the same as FIGS. 12, 13, 14 and 15, however, the counteracting saw blades 150 and 151, were replaced by rows of blades 160 and 161. The slight movement forward-backwards scrapes the nucleus material and again performs the same action of burying the cracker in the nucleus material. Separating side plates 169 and 161 in FIG. 18 performs the nucleus cracking action.

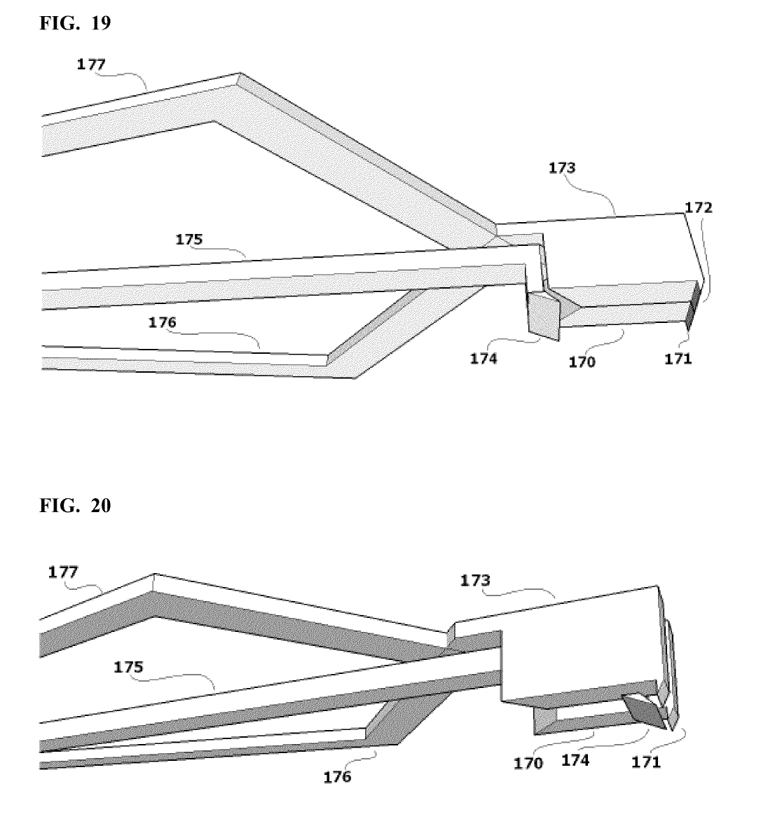

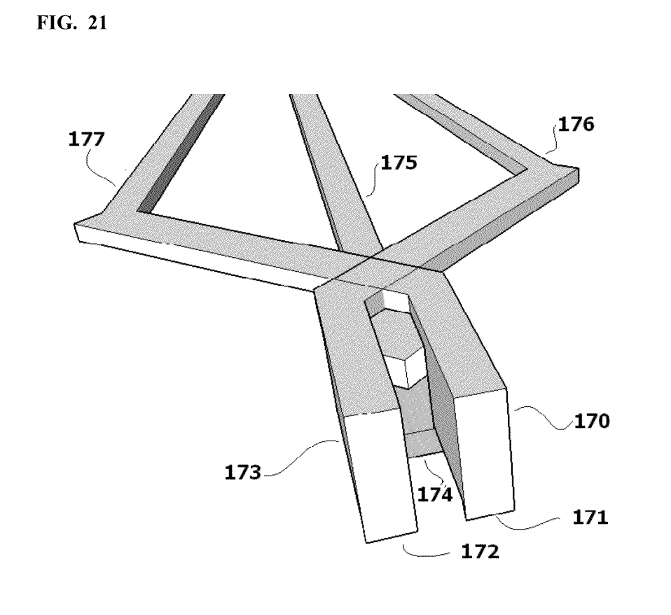

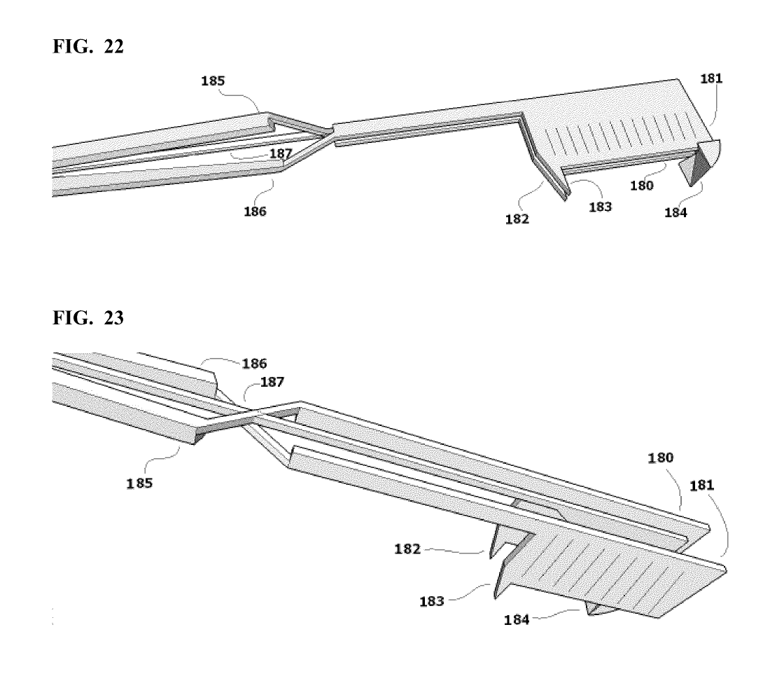

Referring now to FIGS. 19, 20 and 21, the split-incision apparatus shown is a combination of a cracker 170 and 173 and a digger 174 that digs nuclear material under the cracker. In FIG. 19 the two side plates 170 and 173 of the cracker are closed. The cracker portion has blades 171 and 172 that wedge into the nucleus and stabilize the cracker relatively to the nucleus as the digger 174 moves. The digger 174 digs nuclear material under the cracker. In FIG. 20, as the digger move forward, the cracker opens up slightly so that the digger 174 moved forwards. Another illustration is shown in FIG. 21 from a superior view of the split-incision apparatus. In other embodiments, additional side blades or friction surfaces are added to side plates 170 and 173 for further stabilization of the cracker portion of the apparatus in one position relatively to the nucleus as the digger 174 moves beneath it. (Although not shown in FIGS. 19, 20 and 21, an example is shown in FIGS. 22 and 23) When the cracker portion is buried in the nucleus material, the cracker opens up even further. In illustrations FIGS. 19, 20 and 21, cracking action is done by squeezing points 176 against point 177.

Referring now to FIGS. 22 and 23, a different design of the cracker-digger combination is presented where the digger 194 is pulled toward the operator rather than pushed. Wedging blades 182 and 183 of the cracker portion counteracts the digger 184. Again, additional side blades or friction surface shown on surface 181 of FIG. 22 were added to the lateral sides of the cracker for further stabilization of the cracker portion of the apparatus in one position relatively to the nucleus as the digger 184 moves beneath it. FIG. 23 illustrates the separation of plates 180 and 181 by squeezing points 185 against point 186 to perform the nucleus cracking action.

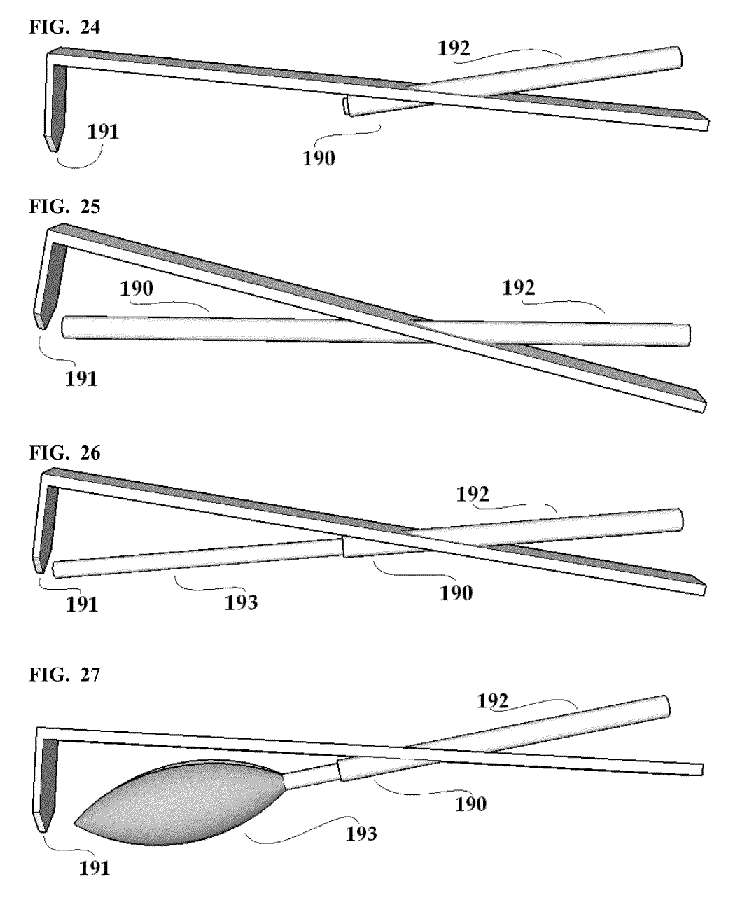

Referring now to FIGS. 24, 25, 26 and 27, another embodiment of the apparatus including a hollow blade 190 for creating a split tunnel in the nucleus counteracted by a wedging blade 191. In other embodiments, the split tunnel is relatively flat on both lateral sides to facilitate a cracking action. As the hollow blade is pulled out, it leaves a cracking balloon 193 that is inflated for the cracking action. Again, in some embodiments, the cracking balloon has relatively flat side walls to facilitate the cracking action. Again, the embodiment shown was drawn as an illustration only as there are many embodiments to accomplish the desired actions. The illustration shown should not constrain to the scope of the concept.

### DESCRIPTION AND OPERATION OF THE EMBODIMENTS

During cataract surgery, the surgeon creates a small entry wound in the periphery of the cornea. A surgeon exposes the lens by tearing the anterior capsule of the lens in a procedure called Capsulorrhexis. After breaking adhesions between the lens and the nucleus by Hydro-dissection, the surgeon insert the new split-incision apparatus through the entry wound.

The split-incision apparatus of FIG. 1 is used to create split-incisions in the nucleus. The surgeon pushes the blades into the nucleus and moves the blades 110 and 111 toward and away from each other. This action creates a split-incision in the nucleus that is used for cracking the nucleus by either bimanual cracking action or by later inserting a cracker device. Variations of the blades like 110-A and 111-A are used to increase the penetration of the blades into the nucleus.

The operation of the split-incision apparatus of FIG. 2 is similar to that of FIG. 1, however the design of the blades removes nuclear material and creates grooves in the nucleus.

The surgeon uses the split-incision apparatus of FIGS. 3, 4, 5 and 6 is able not only to create cuts in the nucleus but also to crack the nucleus by separation of the same blades 120 and 121 in FIG. 3 and blades 130 and 131 in FIGS. 4,5 and 6.

The surgeon using the split-incision apparatus of FIG. 7 creates split-incisions that are perpendicular to the split-incisions made by the split-incision apparatus of FIG. 1. Those split-incisions are also perpendicular the long axis of the split-incision apparatus. This embodiment represents different design of the split-incision apparatus. However, the purpose remains the same; the surgeon uses the device for creating split-incisions in the nucleus for later cracking of the nucleus.

The surgeon using the split-incision apparatus of FIGS. 8 and 9 in similar way to the split-incision apparatus of FIG. 1, however, the male-female configuration of the blades 140 and 141 make insertion through the opening wound safer and the final stage of the cutting is more complete.

The operation of the split-incision apparatus of FIGS. 10 and 11 is somewhat different. The surgeon pushes wedging blade 147 into the nucleus and blades 145 and 146 create two marrow incisions along their way. The left over nucleus material between the incisions is removed by a scooping action or scraping action of blade 147 which is pushed forward together with blades 145 and 146 to remove the nuclear material and to create a wide split-incision.

The operation of the split-incision apparatus of FIGS. 12, 13,14 and 15 is shown. The surgeon moves side plates 150 and 152 back and forth against each other. The lengths of movements of blades 151 against 152 are only a fraction of the length of the blades. The counteracting saws located at the bottom both the two side plates are in contact with an exposed anterior surface of the nucleus. The counteracting action of the saws cut a groove in the nucleus. When the surgeon determine that the side plates were sufficiently buried in the nucleus, the surgeon press on points 154 and 155 to separates side plates 150 and 152 and to crack the nucleus.

Split-incision apparatus of FIGS. 16, 17 and 18 is based on the same principal of split-incision apparatus of FIGS. 12, 13,14 and 15, however, the counteracting saws are replaced by sanders or scrapers 160 and 161.

The surgeon operating split-incision apparatus of FIGS. 12, 13,14 and 15 and split-incision apparatus of FIGS. 16, 17 and 18 may use a separated device attached to the split-incision apparatus to provides manual or motorized power for the counteracting movements of the side plates. This attached device may also assist with the cracking action of the nucleus.

The operation of the split-incision apparatus of FIGS. 19, 20 and 21 is shown. The split-incision apparatus of FIGS. 22 and 23 is based on the same principal of counteraction. In FIGS. 19, 20 and 21, the surgeon moves a digging blade 174 along the lower border of side plates 170 and 173 to remove nuclear material and to create a groove under the side plates 170 and 173. The surgeon used points 176 and 177 to separate side plates 170 and 173 for the cracking action of the nucleus.

Similarly, in FIGS. 22 and 23, the surgeon moves a digging blade 184 to remove nuclear material and to create a groove under side plates 182 and 183.

The operation of the split-incision apparatus of FIGS. 24, 25, 26 and 27 is shown. The surgeon uses the split-incision apparatus to create a split tunnel in the nucleus. After inserting the apparatus to the anterior chamber, the surgeon wedges the wedging blade 191 in the nucleus and pushes hollow blade 190 into the nucleus core. When the Surgeon pulls back the hollow blade 190, the surgeon leaves a cracking balloon in the newly formed split tunnel. Alternatively, a cracking balloon is pushed inside the newly formed split tunnel. At this point, the surgeon inflates the balloon for the cracking action of the nucleus.

After the nucleus is cut and cracked into pieces, the surgeon removes the new split-incision apparatus. Then, the surgeon can proceed with the Phacoemulsification process to remove the pieces of the nucleus from the eye.

### CONCLUSION, RAMIFICATION AND SCOPE

The split-incision apparatus is based on an old principal of counteraction that is applied to create split-incisions in the nucleus. In a number of the embodiments, the new split-incision apparatus further combines creation of split-incisions with a cracking action in the same apparatus.

Thus, while the embodiments of the split-incision apparatus vary considerably, it is to be understood that those descriptions were made by way of example only and were not intended to limit the scope of the present invention. Alterations and further modifications, as well as other and further applications of the principles of the present invention are expected to occur and should not infringe on the rights of the current invention, which is based on the principal of counteracting action to create split-incisions or grooves in the nucleus. The scope of the invention is not limited to structure equivalents as described herein but includes also equivalent structures that perform the recited function and claims of the invention

I claim:

1. A cataract surgical apparatus for creating split incisions in a nucleus of a lens after performing anterior capsulorrhexis, while said nucleus is positioned in its natural capsular bag, comprising:
a. at least two counteracting members, and
b. moving means for moving said two counteracting members toward each other and away from each other along a path, and
c. each of said counteracting members further including cutting means capable of penetrating into said nucleus, and
d. at least one of said cutting means further including at least one cutting edge to cut said nucleus along said path,
whereby said cutting means having said cutting edges are creating split incisions in said nucleus along said path, and
said cutting means without said cutting edges are wedged into said nucleus and have limited movement along said path, and
forces generated by said moving means and applied on said nucleus by said cutting means of one member of said counteracting members are at least partially counteracted by forces applied on said nucleus by other member of said counteracting members.

2. The cataract surgical apparatus of claim 1, wherein said cutting means are shaped to create narrow incisions in said nucleus along said path without breaking off material from said nucleus.

3. The cataract surgical apparatus of claim 1, wherein said cutting means are shaped to break off material from said nucleus and to create wide incisions in said nucleus along said path.

4. The cataract surgical apparatus of claim 1, wherein said cutting means are shaped and angled to divert a vector portion of said forces applied on said nucleus to further deepen said penetration of said cutting means into said nucleus,

5. The cataract surgical apparatus of claim 1
a. wherein outer lateral surfaces of said counteracting members are roughly parallel to each other and at least partially overlapping each other at times when two of said counteracting members are next to each other, and
b. further including separation means to be used at said times for bilateral outward separation of said outer lateral surfaces and inward closing up of said members relatively to said path
whereby at said times and when said lateral surfaces are sufficiently buried in said nucleus, cracking forces are transmitted bilaterally to the sidewalls of said split incisions by said counteracting members and crack said nucleus

6. The cataract surgical apparatus of claim 1, wherein
a. each of said counteracting members further including a side plate in size comparable to side plates of prior art crackers, and
b. said side plates are positioned along said path, and
c. said side plates bottom border are in contact with an exposed anterior surface of said nucleus and roughly vertically to said anterior surface, and
d. said side plates are moved by said moving means forward and backward relatively to each other along said path at a length that is only a fraction of a length of said split incisions length while at least partially overlapping each other, and
e. a plurality of said cutting means having said cutting edges are placed at the bottom border of each of said side plates in direct contact with said nucleus and said plurality of said cutting means of each of said counteracting members are counteracting each other, and
f. said side plates are capable of outward separation bilaterally away from each other and inward closing toward each other, and
g. further including means for outward separation and inward closing toward of said side plates,
whereby said side plates movements forward and backward relatively to each other along said path are acting like two saws in counteracting directions further digging and further breaking off material from said nucleus under said two side plates with each of said movement forward and backward, and further burying said two side plates in said nucleus until sufficiently buried in said nucleus to be used for a cracking action by said side plates separation.

7. The cataract surgical apparatus of claim 1, wherein
a. said one member of said counteracting members further including at least two side plates in size comparable to side plates of prior art crackers, said side plates are positioned along said path, and said side plates bottom border are in contact with an exposed anterior surface of said nucleus, and side plates are roughly vertically to said anterior surface, and said side plates are mostly overlapping each other, and said side plates are capable of outward separation bilaterally to said path away from each other and inward closing of said side plates toward each other, and
b. further including means to outward separation and inward closing of said side plates relatively to each other, and
c. said cutting means of said one member of said counteracting members do not include said cutting edges, and
d. said cutting means of said other member of said counteracting members include said cutting edges and are positioned under the bottom border of said side plates for generating incisions wide enough to allow burying said side plates into said nucleus, whereby a position of said side plates is stabilized relative to said nucleus and said other member breaks off and removes nuclear material under the bottom border of said side plates resulting in said side plates being further buried in said nucleus until sufficient to be separated by said means of outward separation to perform a cracking action on said nucleus.

8. The cataract surgical apparatus of claim 1, wherein said moving means are operated by another device attached to said cataract surgical apparatus to generate said movements either by mechanically transmitter manual power or by motorized power.

9. A cataract surgical apparatus for creating split incisions in a nucleus of a lens positioned in its natural capsular bag opened anteriorly following capsulorrhexis, comprising:
a. two members positioned to penetrate into said nucleus material through an exposed anterior surface of said nucleus, and
b. moving means for generating movement path of said members toward each other and away from each other, and
c. each of said members having at least one penetrating edge for penetrating said nucleus, and
d. at least one member of said two members having at least one side edge for cutting said nucleus along said movement path as said one member is moved relative to said nucleus by said moving means,
whereby said two members penetrate said nucleus by forces applied to push said two members toward said nucleus, and
forces generated by said moving means and transmitted to said members having side edges, which apply said forces on said nucleus, are at least partially counteracted by forces applied on said nucleus by other member of said two members, and
the combined action of said two members creates said split incision in said nucleus along said movement path.

10. The cataract surgical apparatus of claim 9, wherein said side edges are shaped to generate narrow incisions in said nucleus without breaking off material from said nucleus, whereby said split incisions are narrow.

11. The cataract surgical apparatus of claim 9, wherein said side edges are shaped to break off some material from said nucleus and to generate wide incisions in said nucleus, whereby said split incisions are wide.

12. The cataract surgical apparatus of claim 9, wherein said members are shaped to divert a vector portion of said force applied by said moving means on said nucleus to further deepen the penetration of said members into said nucleus.

13. The cataract surgical apparatus of claim 9,
a. wherein outer lateral surfaces of said two members are roughly parallel to each other and at least partially overlapping each other at a times when said two members are next to each other, and
b. further including separation means to be used at said times for bilateral outward separation of said members and inward closing up of said members relatively to said movement path
whereby at said times and when said lateral surfaces are sufficiently buried in said nucleus, cracking forces are transmitted bilaterally to the sidewalls of said split incisions by said members and crack said nucleus.

14. The cataract surgical apparatus of claim 9,
a. wherein each of said two members further including side plate in size comparable to side plates of prior art crackers, and said two side plates are positioned along said movement path, and both side plates bottom border are in contact with said exposed anterior surface of said nucleus and roughly vertical to said exposed anterior surface of said nucleus, and
b. said side plates are moved by said moving means forward and backward relatively to each other along said movement path at a length that is only a fraction of a length of said split incisions length while keeping said side plates at least partially overlapping each other, and
c. a plurality of said penetrating edges and said side edges are situated on said bottom border of each of said side plates and said penetrating edges and said side edges of each member are counteracting forces of said penetrating edges and said side edges of a remainder member of said two members, and
c. said side plates are capable of bilateral outword separation from each other relative to said movement path and inward closing toward each other
d. further including means for outward separation of and inward closing of said side plates,
whereby said side plates moving forward and backward relatively to each other are acting like two saws moving in counteracting directions further digging and breaking off material from said nucleus under said two side plates with each of movement of said side plates, burying said two side plates in said nucleus until sufficiently buried in said nucleus to be used for a cracking action by said side plates separation means.

15. The cataract surgical apparatus of claim 9,
a. wherein said other member of said two counteracting members further including at least two side plates in size comparable to side plates of prior art crackers, said side plates are positioned along said movement path, and both side plates bottom border are in contact with said exposed anterior surface of said nucleus and roughly vertically to said exposed anterior surface and said side plates are mostly overlapping each other, capable outward separation bilaterally to said movement path away from each and inward closing of said side plates toward each other, and
b. further including means to outward separation and inward closing of said side plates relatively to each other, and
c. said cutting means of said other member of said two counteracting members do not have cutting edge and just wedge into said nucleus material, and
d. said cutting means of said one member of said two counteracting members is moved by said moving means and said cutting means are capable of breaking off material of said nucleus under said bottom border of said side plates, and to generate wide split incisions in said nucleus under said bottom border of said side plates,
whereby said side plates are stabilized related to said nucleus and said one member of said two counteracting members removes nuclear material under the bottom border of said side plates of said other member resulting in said side plates being further buried in said nucleus until sufficient to be separated by said means of outward separation and perform a cracking action of said nucleus.

16. The cataract surgical apparatus of claim 9, wherein moving means are propel by another device capable to be attached to said cataract surgical apparatus and generate said movements either by mechanically transmitter manual power or by motorized power.

17. A cataract surgical apparatus for creating split tunnels and cracking a nucleus of a lens positioned in its natural capsular bag opened anteriorly by a capsulorrhexis procedure, comprising:
a. an elongated member having wedging means for wedging into said nucleus and stabilizing said elongated member position relatively to the nucleus, and
b. a hollow blade capable of penetrating said nucleus into its core for creation of split tunnels in said nucleus, and
c. moving means capable of moving said hollow blade into said nucleus and out of said nucleus, and
d. an inflatable means design to pass through said hollow blade into said split tunnels,
whereby forces applied on said nucleus by said hollow blade penetrating said nucleus core are at least partially counteracted by forces applied on said nucleus by said elongated member wedged into said nucleus, and
inflating said inflatable means in said split tunnel causes cracking of said nucleus.
